(19) United States
(12) Patent Application Publication
(10) Pub. No.: US 2009/0182216 A1
(43) Pub. Date: Jul. 16, 2009

(54) MOISTURE CONTROL IN A TRANSDERMAL BLOOD ALCOHOL MONITOR

(76) Inventors: William James Roushey, III, Littleton, CO (US); Jeffrey Scott Hawthorne, Bennett, CO (US); Mark Henry Wojcik, Littleton, CO (US); David Cain Collins, Denver, CO (US); Steven Keith McGee, Lakewood, CO (US)

Correspondence Address: STANLEY J. GRADISAR, 1182 THATCH CIRCLE, CASTLE ROCK, CO, 80109, US

(21) Appl. No.: 12/013,931

(22) Filed: Jan. 14, 2008

(51) Int. Cl. A61B 5/00

(52) U.S. Cl. 600/364

(57) ABSTRACT

Moisture may build up inside an alcohol monitor that is securely attached to a human subject due to the inlet air from the subject's skin surface which constantly emits water vapor in the form of insensible skin perspiration. As the warm moist air which has very high humidity flows along the air flow path through decreasing temperatures within the alcohol monitor, moisture will be removed from the air through condensation. The condensation problem is solved by lowering the humidity level in the air sample by mixing the very humid air sample from the body with less humid ambient air, which increases the dew point for condensation. Increasing the dew point in the air sample means that there must be a greater change in temperature along the air flow path of the air sample in order to cause the moisture in the air sample to condense and become water.

### CROSS-REFERENCES TO RELATED APPLICATIONS

Reference is made to U.S. Pat. No. 5,220,919 titled “BLOOD ALCOHOL MONITOR,” European Patent No. EP 0623001 B1 titled “BLOOD ALCOHOL MONITOR,” and to the following co-pending applications: U.S. patent application Ser. No. 10/441,940 titled “METHOD AND APPARATUS FOR REMOTE BLOOD ALCOHOL MONITORING,” U.S. patent application Ser. No. 11/411,692 titled “METHOD AND APPARATUS FOR REMOTE BLOOD ALCOHOL MONITORING,” U.S. patent application Ser. No. 11/411,694 titled “METHOD AND APPARATUS FOR REMOTE BLOOD ALCOHOL MONITORING,” U.S. patent application Ser. No. 11/411,686 titled “METHOD AND APPARATUS FOR REMOTE BLOOD ALCOHOL MONITORING,” and U.S. patent application Ser. No. 11/454,491 titled “MOISTURE CONTROL IN A TRANSDERMAL BLOOD ALCOHOL MONITOR,” all owned by the assignee of this invention and all are incorporated herein by reference in their entirety for all that is taught and disclosed therein.

### TECHNICAL FIELD

This invention relates to transdermal blood alcohol monitors for continuous monitoring of blood alcohol levels, and more particularly, the invention relates to improved moisture control within a transdermal blood alcohol monitor or similar device.

### BACKGROUND

Individuals on probation, parole, or in alcohol treatment programs may be prohibited from consuming alcohol, and many federal, state, and local law enforcement agencies require testing to ensure participants in court ordered programs remain alcohol free. In general, present-generation remote alcohol monitoring devices used in probation, parole, and treatment settings are fixed-location breath-testing devices that measure Blood Alcohol Content (“BAC”) and incorporate voice or video identification of the participant. If a subject tests positive for alcohol, the monitoring device then sends a message alerting the monitoring center of a violation by the subject, and the monitoring center then sends an alert message to the subject's supervising agency or dedicated administrator.

As alcohol is ingested orally, it is absorbed into the body's blood and distributed throughout the body via the circulatory system. Alcohol is eliminated from the body by two mechanisms: metabolism and excretion. Metabolism accounts for the removal of greater than 90% of the alcohol consumed, removing it from the body via oxidation of the ethyl alcohol molecule to carbon dioxide and water primarily in the liver. The remaining alcohol is excreted unchanged wherever water is removed from the body—breath, urine, insensible skin perspiration, and saliva. Although excretion accounts for less than 10% of the eliminated alcohol, it is significant because unaltered alcohol excretion permits an accurate measurement of alcohol concentration in the body by way of both breath analysis and insensible skin perspiration. Insensible skin perspiration is the vapor that escapes through the skin through sweating. The average person will emit approximately one liter of insensible skin perspiration each day. This insensible skin perspiration can be used to obtain a transdermal measurement of blood alcohol concentration, referred to as Transdermal Alcohol Concentration (“TAC”).

Transdermal monitoring of blood alcohol levels is accomplished by taking percentage measurements of alcohol contained in the insensible skin perspiration that is expelled transdermally through human skin. Throughout this description of the invention, insensible skin perspiration may be referred to as “vapor,” “air vapor,” “air vapor sample,” “air vapor volume,” “sample,” “sample volume,” “air sample,” and “air sample volume,” interchangeably, with no difference in meaning intended. A monitoring device is attached to the skin to capture the air vapor and measure the alcohol contained therein, if any.

There are numerous advantages to transdermal alcohol monitoring, as opposed to breath-testing, including, but not limited to, the ability to take readings at any time without the knowledge of the subject, consistent and continuous testing (unlike breath alcohol testing where a subject breathing incorrectly into the testing device can cause inaccurate results), and the ability to convert such readings into electrical signals that can be transmitted to a central monitoring station.

However, there is a need to better manage the build-up of moisture within a transdermal blood alcohol monitor to prevent damage to the various internal components, and to increase the service life of the transdermal blood alcohol monitor. The present invention meets these and other needs in the art.

### BRIEF DESCRIPTION OF THE DRAWINGS

FIG. 1 shows a block diagram of an alcohol monitoring system of the present invention.

FIG. 2A shows a cross-section view and FIG. 2B shows a perspective view of the alcohol monitor of the present invention.

FIGS. 3A and 3B show exploded perspective views of the alcohol monitor of the present invention.

FIG. 4 shows an exploded perspective view of a disposable cartridge of the alcohol monitor of the present invention.

FIG. 5 shows an exploded perspective view of an inner sample system assembly of the alcohol monitor of the present invention.

FIG. 6 shows the air flow path of the air sample through an exploded perspective view of the various air flow path components of the alcohol monitor of the present invention.

FIG. 7 shows the air flow path and the relationship between gravity and the direction of the air sample flow through the alcohol monitor of the present invention.

### DETAILED DESCRIPTION

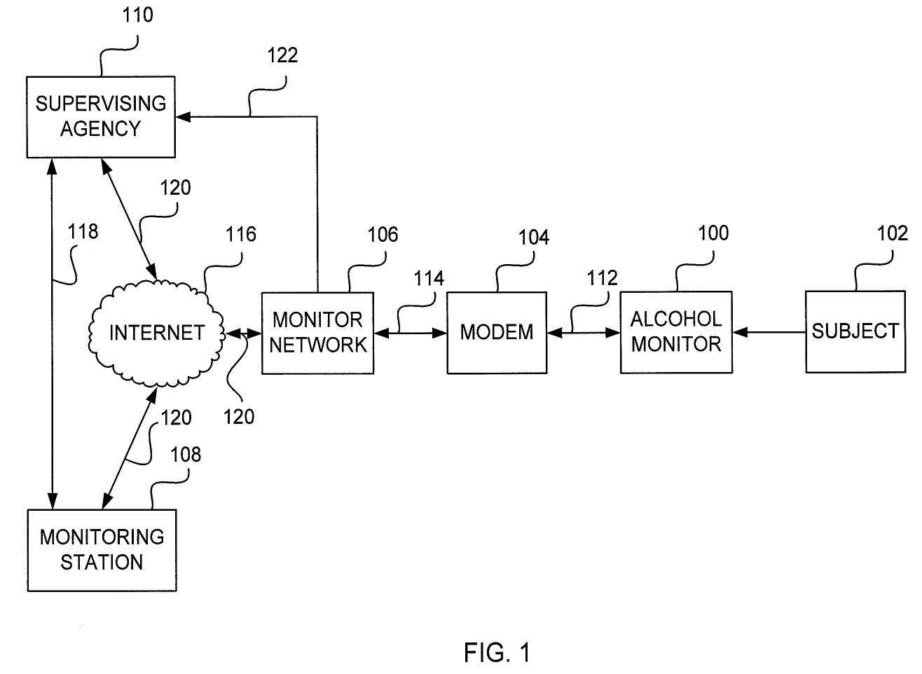

Referring now to the Figures, in which like reference numerals refer to structurally and/or functionally similar elements thereof, FIG. 1 shows by way of illustrative example a system block diagram of remote blood alcohol monitoring between a human Subject 102 and a Monitoring Station 108 utilizing Alcohol Monitor 100 of the present invention. In one embodiment, Alcohol Monitor 100 weighs about eight ounces, is waterproof, designed to handle the stress of everyday activity, and can be worn under any conditions, including bathing and swimming. Alcohol Monitor 100 is attached to the Subject 102. Once Alcohol Monitor 100 is in place, it cannot be removed without triggering a tamper alarm, which is recorded in Alcohol Monitor 100. In addition, there are a number of anti-tamper features designed into Alcohol Monitor 100 to ensure that the TAC readings taken are from Subject 102, and accurately represent the blood alcohol level of Subject 102 and not some other person. Though this discussion focuses on one Subject 102, one skilled in the art will recognize that many Alcohol Monitors 100 may be attached to many Subjects 102 at the same time over a broad geographic area, and all may be monitored by Monitoring Station 108, which is the intended purpose. Likewise, there may be multiple Monitor Networks 106 and Monitoring Stations 108 that manage additional Subjects 102 in diverse geographic locations.

Alcohol Monitor 100 will take TAC readings that are time stamped at predetermined or random intervals twenty-four hours a day, seven days a week, 365 days a year, without active participation by Subject 102. Testing schedules may range from as frequent as every 30 minutes or as infrequent as once per day. Alcohol Monitor 100 collects TAC readings from Subject 102 regardless of the location or activity of Subject 102. While commuting, at work, at home, during recreation, in the shower, or sleeping, Subject 102 is passively monitored, allowing for continual, effective monitoring while Subject 102 maintains a normal routine. Subject 102 typically does not know when the sampling will occur. Typical existing alcohol monitoring programs that have used other means of testing subjects for alcohol will likely see an increase in the number of program positives utilizing the present invention. This is a result of the continuous monitoring, rather than the pre-arranged, specific testing times typical of current monitoring programs. Continuous monitoring eliminates the ability for subjects to manipulate their drinking patterns to avoid detection.

TAC readings are taken as scheduled without the participation of Subject 102, with the data uploaded at scheduled time intervals to a Modem 104, or immediately if a positive drinking event or a tamper is detected and Modem 104 is in range. Typically, Modem 104 would be placed at the residence of Subject 102, and Subject 102 is merely required to periodically be in proximity to Modem 104 for the purpose of allowing automatic transmission of TAC readings taken by Alcohol Monitor 100 over a period of time. Subject 102 comes within range of Modem 104, typically within about ten to twenty feet, on a periodic basis, such as once per day, to allow the automatic transmission to take place. Different hardware components may increase or decrease the range at which the automatic transmission will take place. Subject 102 may rise and leave for work, return home, and remain at home until the next day when it is time to leave for work again. When Alcohol Monitor 100 is in range and the timer indicates that it is time to communicate with Modem 104, Alcohol Monitor 100 will transfer to Modem 104 through radio frequency (“RF”) signals through bi-directional RF Communication Link 112 all the TAC readings, tamper indicators, error indicators, diagnostic data, and any other data stored in Alcohol Monitor 100 regarding Subject 102. Modem 104 also can transmit operational information, such as monitoring schedules and reporting schedules in the form of RF signals back to Alcohol Monitor 100 over bi-directional RF Communication Link 112.

Modem 104 stores the data contained in the RF signals received from Alcohol Monitor 100 for transmission to Monitor Network 106. After receiving all of the information from Alcohol Monitor 100, Modem 104 will check the stored data for any TAC readings, tampers, errors, or diagnostic data. Any one of these, or a trigger from a predetermined time interval, will cause Modem 104 to establish a connection over a Communication Link 114 with Monitor Network 106. Once a connection is established, Monitor Network 106 validates the identity of Modem 104 and authenticates the data before it is stored. Once validated, Modem 104 will transfer all of the TAC readings, tampers, errors, diagnostic data, and any other data stored to a web-hosted database server at Monitor Network 106 where all data is permanently stored. Monitor Network 106 then analyzes the data received and separates and groups the data into a number of separate categories for reporting to monitoring personnel at Monitoring Station 108. The data can then be accessed by the monitoring personnel through the use of secured dedicated websites through Internet 116 and an Internet Connection 120 to Monitor Network 106. When Monitor Network 106 analyzes the data received, an automatic alert, based upon a rules-based database, may be sent directly from Monitor Network 106 to a call center at a Supervising Agency 110 over a Communication Link 122, or to an individual previously designated by Supervising Agency 110, when a specific alert, or combination of alerts, are received. The alert may be an e-mail, a fax, or a page to a previously provided number. Communication Link 122 may be a wire or wireless connection.

Monitor Network 106 may be located at Monitoring Station 108, or in a separate location. Monitoring personnel at Monitoring Station 108 have access to all of the data gathered on all of the Subjects 102. Supervising personnel at the call center of Supervising Agency 110, however, only have access to those Subjects 102 that are associated with Supervising Agency 110.

Monitoring Station 108 may automatically or periodically transmit data received from Modem 104 via Monitor Network 106 to one or more persons at Supervising Agency 110 who are assigned to monitor Subject 102, such as a parole officer, probation officer, case worker, or other designated person or persons in charge of enrolling Subject 102 and monitoring the data being collected on Subject 102. Only one Supervising Agency 110 is shown for simplicity, but one skilled in the art will recognize that many Supervising Agencies 110 may be accessing Monitor Network 106 at any given time. A connection is established with Supervising Agency 110 through a Communication Link 118. Typically this connection is accomplished via the telephone system through a wire or wireless link, and may connect to a pager or cellular phone of the designated person. Designated personnel at Supervising Agency 110 may also access Monitor Network 106 through the use of secured dedicated websites through Internet 116 and Internet Connection 120 to Monitor Network 106. Monitor Network 106 web software allows Supervising Agency 110 the ability to track Subject 102 compliance in a manner most feasible to them, and can be defined to fit the needs of both small and large programs. Each Supervising Agency 110 may customize the frequency of monitoring and the method of notification for alerts that they want to receive from Monitor Network 106. Alerts may be categorized by the type and severity of alert, allowing each Supervising Agency 110 to prioritize and better categorize a response (i.e., a low battery warning versus a possible alcohol violation).

Each Supervising Agency 110 has its own separate data storage area on the database server at Monitor Network 106 so that representatives from each Supervising Agency 110 can retrieve the secure data they need when they need it. Existing monitoring agencies that are experienced at managing alcohol offenders may easily take advantage of this approach.

Utilizing Alcohol Monitor 100 with the system described has many advantages and benefits over existing methods and apparatus, including, but not limited to, no collection of body fluids (blood, breath, urine) that require special gathering, handling, or disposal considerations; no waiting for laboratory test results; there is no need for the subject to travel to a test center; continuous 24/7/365 monitoring and data collection from any location; no subject, agency official, or laboratory intervention—only passive participation on the part of the subject; the monitoring device is light weight and can be hidden from normal view; tamper-resistant technology ensures accurate readings representative of the subject being monitored; advanced technology utilizing microprocessors, encrypted data links, and secure data storage and retrieval; the ability for monitored subjects to maintain normal daily routines, including work, counseling, community service, family obligations, and recreation; and easy, web-based, secure access for the monitoring agency to each subject's data.

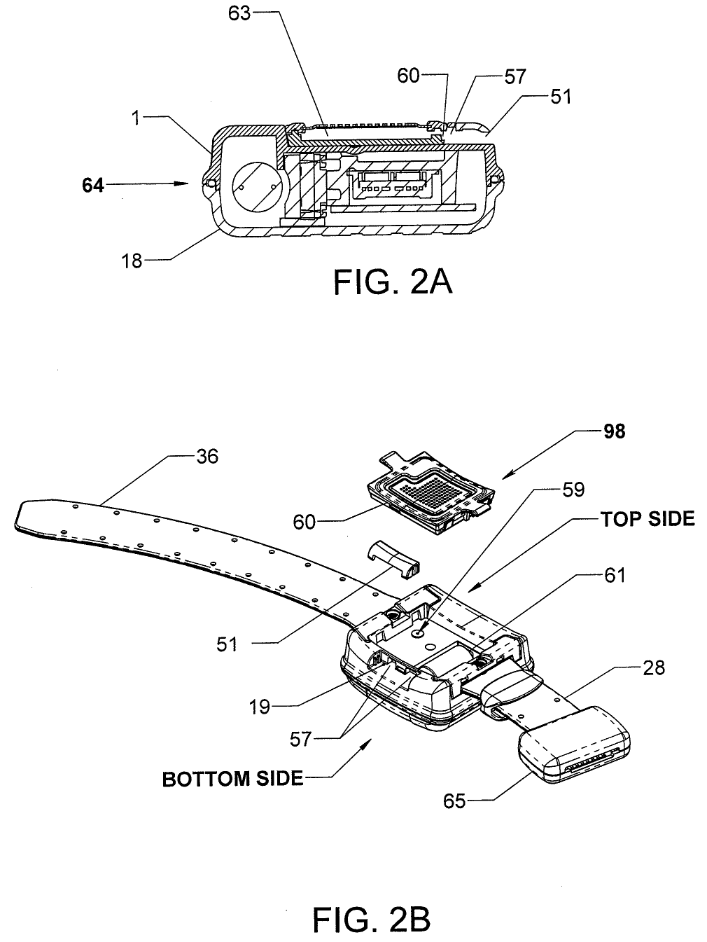

Referring now to FIGS. 2A and 2B, an embodiment of Alcohol Monitor 100 is illustrated for attachment to a human Subject 102. Alcohol Monitor 100 is in the form of a bracelet broadly comprised of a Housing 64 which is assembled from a Outer Housing 1 and an Inner Housing 18, attached to a First Conductive Strap 28 terminated with a Securing Buckle 65 and a Second Conductive Strap 36 attached to the opposite side of Housing 64, which will fit through Securing Buckle 65, all of which enable the bracelet to encircle the limb of a human Subject 102, such as an arm or a leg. Housing 64 also contains a Battery 61, a Disposable Cartridge 98 that snaps into Outer Housing 1, and lines up with a Cartridge Outlet 59. Disposable Cartridge 98 may be replaced from time-to-time. Disposable Cartridge 98 forms a Sample Collection Chamber 63 as well as a Cartridge Vent 60 that allows ambient air to enter Sample Collection Chamber 63. Cartridge Vent 60 is sized small enough and oriented on Housing 64 so that water will not enter into Sample Collection Chamber 63. Housing 64 also contains a set of Moisture Drains 57 which allow any water that may collect around Disposable Cartridge 98 to drain out of Housing 64. Though Moisture Drains 57 and the Cartridge Vent 60 are both partially covered up by an Exhaust Cover 51, Moisture Drains 57 and Cartridge Vent 60 are separate from one another. The entire Housing 64 is designed so that when worn by Subject 102 in an upright walking or standing position, gravity will help drain any moisture out of Housing 64 through the Moisture Drains 57. Thus, Housing 64 has a TOP SIDE and a BOTTOM SIDE when worn by Subject 102 as shown in FIG. 2B.

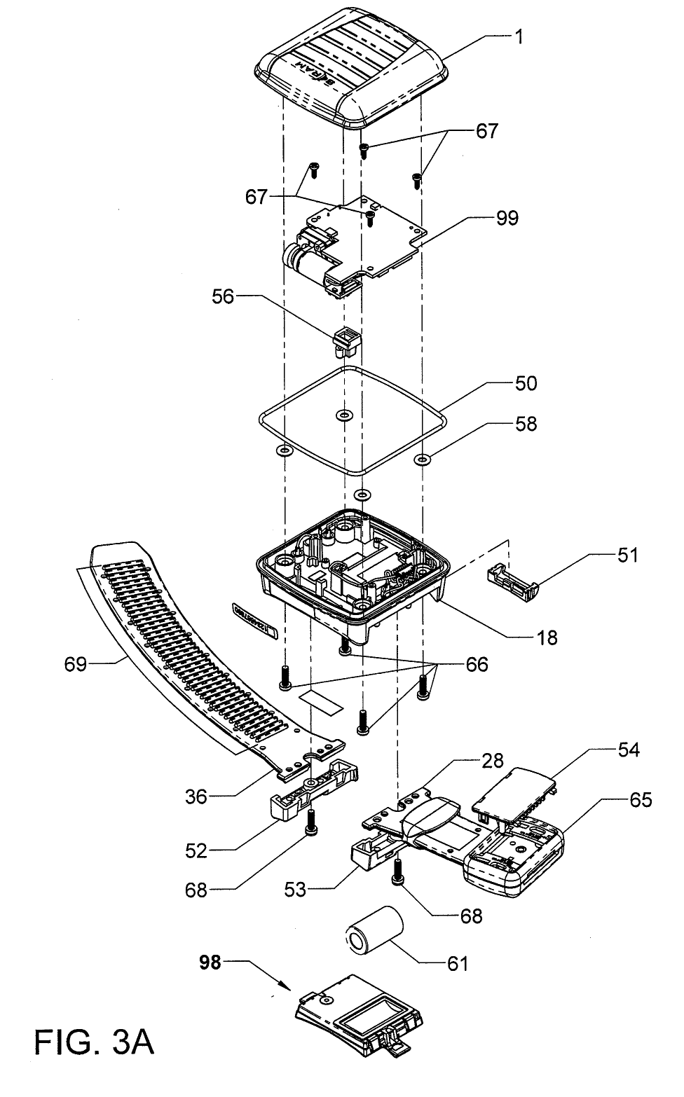

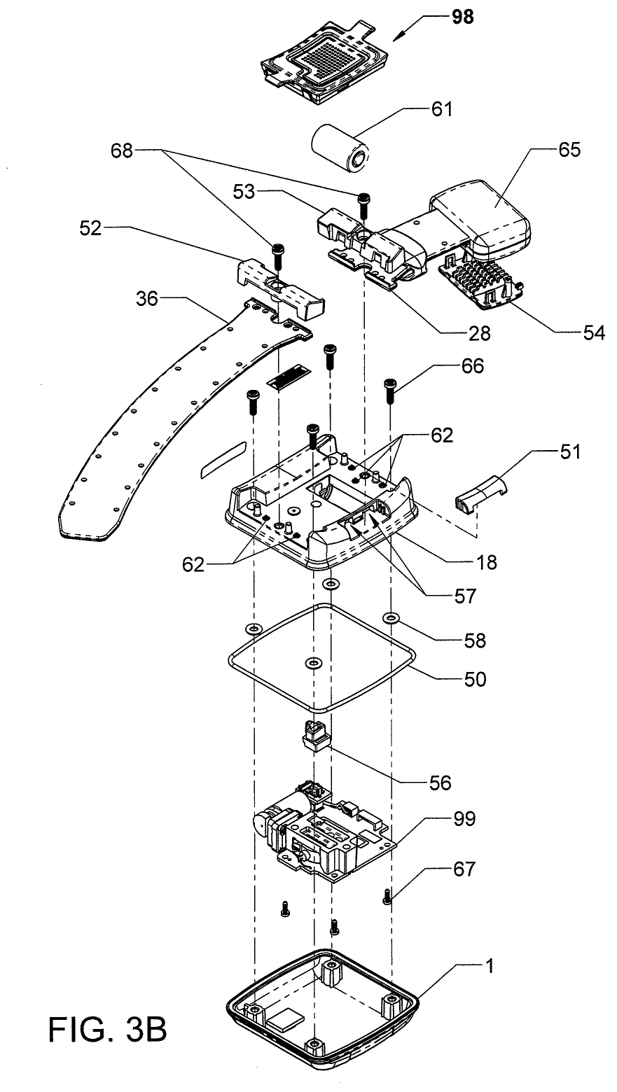

Referring now to FIGS. 3A and 3B, Housing 64 (see FIG. 2) consists of Inner Housing 18 with a Case Sealing O-Ring 50 which provides a water proof seal between Inner Housing 18 and Outer Housing 1. Four Screws 66 are inserted through Inner Housing 18 and through individual O-Rings 58, and into Outer Housing 1. Encased within Inner Housing 18 and Outer Housing 1 is an Inner Sample System Assembly 99, which is secured to Inner Housing 18 with four Screws 67. There is also an Infrared Shield 56 which is used to shield an infrared tamper sensor from ambient light. Second Conductive Strap 36 is fastened to Inner Housing 18 and makes contact with a series of Pogo Pins 62 which will allow for an electrical connection from an electrical circuit board and Second Conductive Strap 36. Second Conductive Strap 36 is fastened permanently in place with a Cover Plate 52 and a screw 68. First Conductive Strap 28 is also fastened to Inner Housing 18 and makes contact with another series of Pogo Pins 62 which allow for an electrical connection from the electrical circuit board and First Conductive Strap 28. First Conductive Strap 28 is fastened permanently in place with a Cover Plate 53 and another Screw 68. Once Housing 64 is assembled, Exhaust Cover 51 is snapped in place to partially cover Moisture Drains 57 and Cartridge Vent 60.

Second Conductive Strap 36 has a series of Ridges 69 aligned in the center of it which are designed to fit in cooperation with Securing Buckle 65 so that Alcohol Monitor 100 may be adjustably tightened to fit securely to a limb of Subject 102. A Securing Cover Plate 54 is affixed to Securing Buckle 65 which holds the straps permanently in place and keeps Subject 102 from tampering with the straps in an attempt to remove Alcohol Monitor 100.

Once fixed in place, it is impossible to remove Alcohol Monitor 100 from a limb of Subject 102 without cutting First Conductive Strap 28 or Second Conductive Strap 36, or otherwise breaking Securing Cover Plate 54 or Securing Buckle 65. When it becomes necessary to replace Battery 61, or simply to remove Alcohol Monitor 100 from Subject 102, Securing Cover Plate 54 on Securing Buckle 65 must be broken loose and replaced. Battery 61 can be replaced by removing Disposable Cartridge 98.

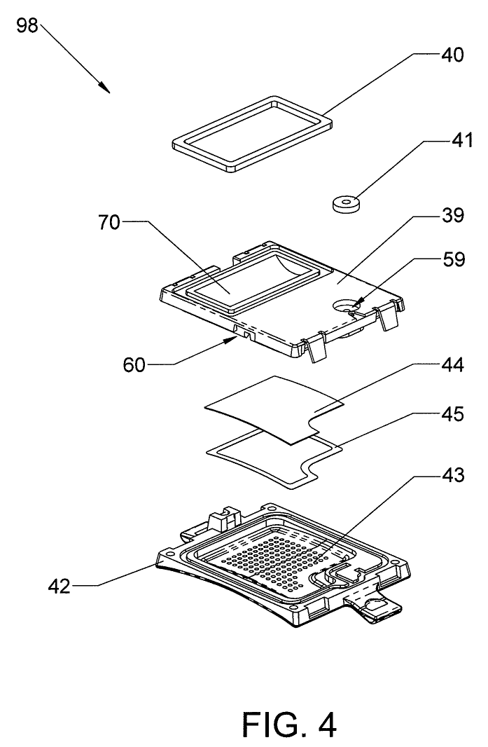

Referring now to FIG. 4, Disposable Cartridge 98 is assembled from an Inlet Plate 43, which is inserted into a Cartridge Front 42 which makes up the front of Disposable Cartridge 98. An Adhesive Gasket 45 is laid on top of Inlet Plate 43. A water resistant (hydrophobic) Gore Membrane 44 is placed on top of Adhesive Gasket 45 which adheres Gore Membrane 44 to Inlet Plate 43. This assembly creates a water resistant barrier that allows air to pass through it but not water. Sample Collection Chamber 63 (see FIG. 2A) is completed by ultrasonically welding the Cartridge Back 39 to Cartridge Front 42. Cartridge Vent 60 is a small hole located in Cartridge Back 39. Cartridge Vent 60 allows ambient air to enter into Sample Collection Chamber 63. This ambient air lowers the humidity level of the insensible perspiration sample that is being collected in Sample Collection Chamber 63. Disposable Cartridge 98 contains a small exit hole, Cartridge Outlet 59, which allows the sample air collected in Sample Collection Chamber 63 to be moved out of Disposable Cartridge 98. Cartridge Outlet 59 is sealed by a Cartridge Gasket 41. There is also an Indentation 70 in Cartridge Back 39, which becomes the cover for Battery 61. The battery compartment is sealed by a Battery Door Gasket 40.

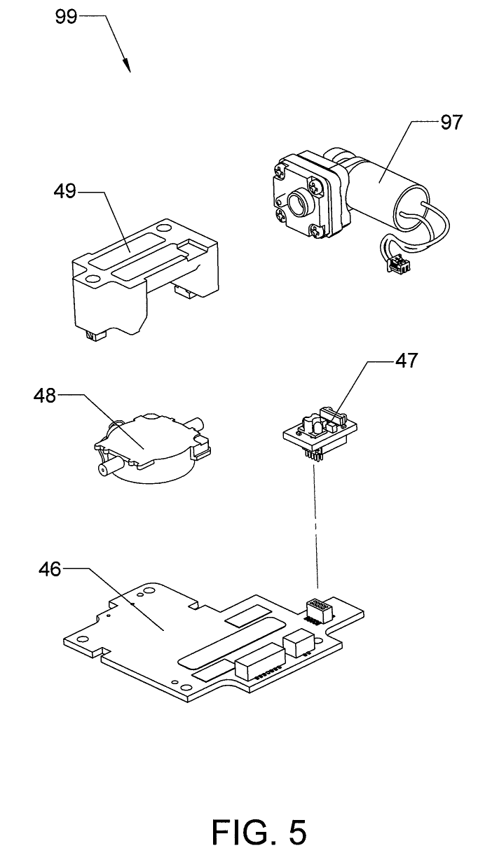

Referring now to FIG. 5, Inner Sample System Assembly 99 is an assembly of five separate components. The base of the assembly is a Main Printed Circuit Board Assembly (Main PCBA) 46. A Secondary Printed Circuit Board Assembly (Secondary PCBA) 47 plugs into Main PCBA 46. A Fuel Cell 48 mounts on top of Main PCBA 46 and is covered by a Fuel Cell Grommet 49. Fuel Cell Grommet 49 provides an interface from Pump Assembly 97 to Cartridge Outlet 59 where the air sample leaves Disposable Cartridge 98, and from Fuel Cell 48 to Exhaust Vent 19 where the air sample leaves Housing 64. Fuel Cell Grommet 49 fits over the top of Fuel Cell 48 and then snaps into groves built into Main PCBA 46, thus securing Fuel Cell 48 to Main PCBA 46. Pump Assembly 97 plugs into the end of Fuel Cell Grommet 49.

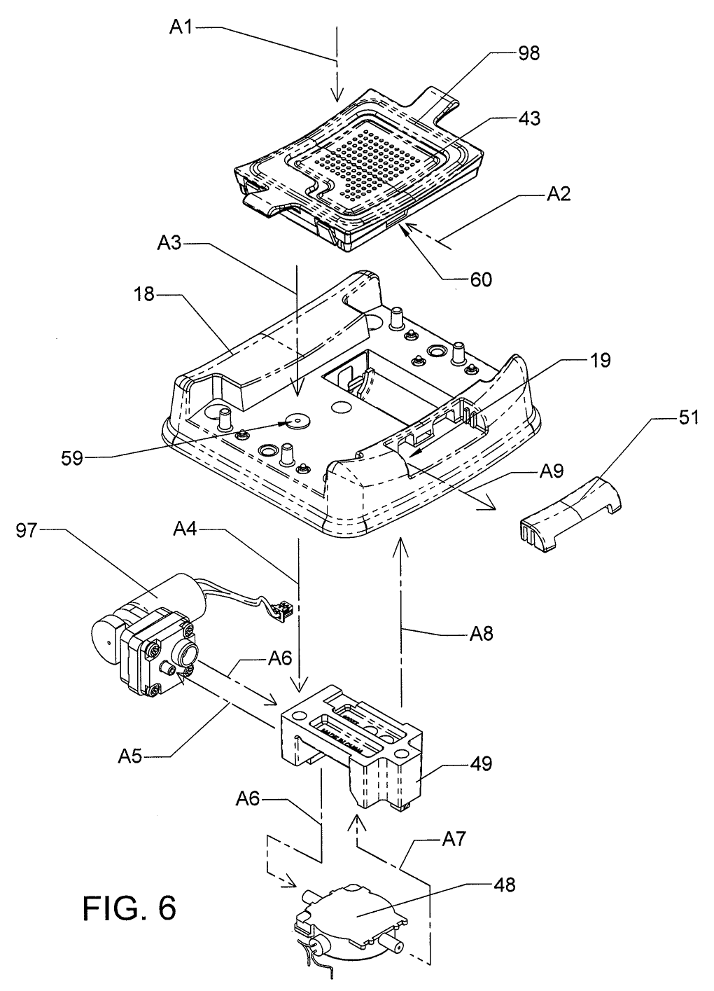

Referring now to FIG. 6, an air flow path is described and indicated by arrows A1-A9. An air vapor sample (A1) is moved into Disposable Cartridge 98 from the skin of Subject 102 through Inlet Plate 43 by Pump Assembly 97. Ambient air is moved into (A2) Disposable Cartridge 98 through Cartridge Vent 60 in the side of Disposable Cartridge 98 by Pump Assembly 97. It should be noted that it is possible to operate Alcohol Monitor 100 without Pump Assembly 97 as long as the air flow path has no physical barriers. Since there is a fairly constant flow of insensible skin perspiration out from the skin of Subject 102, there is a positive force for moving the insensible skin perspiration through the air flow path once Alcohol Monitor 100 is attached to a limb of Subject 102. However, it has been found to be greatly advantageous to utilize Pump Assembly 97 to control the amount of air vapor sample that is passed through Fuel Cell 48, and for drawing in ambient air along with the air vapor sample from the skin of Subject 102 into Sample Collection Chamber 63 of Disposable Cartridge 98.

Pump Assembly 97 draws the combined air sample (air vapor sample and ambient air) (A3) from Sample Collection Chamber 63 of Disposable Cartridge 98 through Cartridge Outlet 59 located in Inner Housing 18. The combined air sample is then moved (A4) into Fuel Cell Grommet 49 and into Pump Assembly 97 (A5), then moved back out of Pump Assembly 97 (A6) through Fuel Cell 48, where the combined air sample passes across the face of Fuel Cell 48 generating a TAC reading, and into Fuel Cell Grommet 49 (A7). The combined air sample is moved (A8) back into Inner Housing 18 and then exits Inner Housing 18 through Exhaust Vent 19 located under Exhaust Cover 51 (A9) and to the ambient air outside of Housing 64.

In order for Alcohol Monitor 100 to reliably measure blood alcohol content, the insensible skin perspiration which is emitted from the body in the form of air vapor will migrate away from the skin and through Inlet Plate 43 and into Disposable Cartridge 98 of Housing 64. These air vapors collect in Sample Collection Chamber 63 located in Disposable Cartridge 98 where it mixes with ambient air that is let in through Cartridge Vent 60. Pump Assembly 97 is activated to draw the combined air sample from Disposable Cartridge 98, through Cartridge Outlet 59 into Fuel Cell Grommet 49, and into Pump Assembly 97. The air sample is then moved out of Pump Assembly 97 through Fuel Cell 48 into Fuel Cell Grommet 49, where it passes into Inner Housing 18 and out of Exhaust Vent 19.

In order to avoid false readings, it is important that Alcohol Monitor 100 be waterproof to prevent the entry of water directly into the air flow path. It is also important that any moisture in the air sample itself be removed, and any water condensation resulting from temperature changes between the point where the air sample enters into Alcohol Monitor 100 to the point where sensor measuring takes place is eliminated or minimized.

A problem encountered with transdermal blood alcohol monitors, such as the transdermal blood alcohol monitor described in U.S. patent application Ser. No. 10/441,940, (hereinafter referred to as the “940 alcohol monitor”), is moisture build up along the air flow path beginning from the inlet into the alcohol monitor next to the skin of the subject, through the interior of the alcohol monitor, and exiting through the exhaust port. Moisture buildup inside an alcohol monitor is understandable, given that the source of the inlet air is directly from the subject's skin surface, which constantly emits water vapor in the form of insensible skin perspiration. The rate at which moisture builds up inside an alcohol monitor depends in part upon the subject, as each person has a varying amount of perspiration that their body gives off. Condensation of moisture into water droplets within an alcohol monitor can eventually damage internal components, thus reducing the service life of the alcohol monitor. When water buildup is too great within an alcohol monitor, the water may prevent alcohol readings from being taken. This is because alcohol is water soluble, and the fuel cell sensor will not sense the alcohol suspended in water. Alcohol Monitor 100 of the present invention solves these water condensation problems associated with prior alcohol monitors.

Laboratory studies have shown that the sample inlet chamber of the '940 alcohol monitor reaches a relative humidity level as high as 95% within the first twenty-four hours of wear by the subject. This high humidity level, along with normal variations in ambient air temperature, creates an environment inside the air flow path that promotes water condensation. In a closed system with 95% humidity, the dew point is within a couple of degrees of the air temperature within the closed system. Dew point temperature is defined as the temperature to which the air would have to cool (at constant pressure and constant water vapor content) in order to reach saturation. A state of saturation exists when the air is holding the maximum amount of water vapor possible at the existing temperature and pressure. When the dew point temperature and air temperature are equal, the air is said to be saturated. If the relative humidity is 100%, the dew point will be equal to the current temperature. As relative humidity falls, the dew point becomes lower, given the same air temperature. Dew point temperature is never greater than the air temperature.

Therefore, if the humidity level in the '940 alcohol monitor is at or near 95%, and there is a temperature difference from four or five or more degrees C. from the air inlet at the inlet plate next to the skin to the fuel cell sample chamber, moisture will be removed from the air through condensation along the air flow path as the warm moist air flows through decreasing temperatures. With each degree drop in temperature, there will be more and more condensation along the air flow path, forming tiny droplets of water within the '940 alcohol monitor.

Alcohol Monitor 100 of the present invention solves this water condensation problem by first simplifying the air flow path by eliminating many of the physical barriers that trap and retain moisture. Second, additional changes made to the air flow path take advantage of gravity, allowing any water droplets that form to flow out of Alcohol Monitor 100 while the subject is in an upright position (walking or standing). Third, by allowing ambient air to enter Disposable Cartridge 98 the humidity level will be lowered from 95% in Sample Collection Chamber 63 to approximately 30%, which is fairly constantly maintained along the air flow path, thereby lowering the dew point temperature causing the moisture in the sample to continue to be held in it's vapor state. Thus the elimination of potential moisture condensation internal to Alcohol Monitor 100 is achieved.

Many of the separate chambers found in the '940 alcohol monitor have been eliminated from Alcohol Monitor 100 in order to achieve a simpler air flow path. Once water droplets formed in the '940 alcohol monitor, they became trapped between the multiple membranes contained therein. Water at times was pulled into the pump, forced out, and sprayed into the fuel cell chamber. The membrane on the exit port also trapped moisture. All of this trapped moisture over time tended to damage various internal components. Trapped moisture caused corrosion on electrical components and on the pump. Corrosion buildup on the pump eventually causes failure. Moisture also caused corrosion on the contacts for the flex circuit connector, eventually causing an electrical failure. Instead of having multiple membranes, forming essentially multiple chambers, Alcohol Monitor 100 of the present invention only has a single membrane located internally along the air flow path, Gore Membrane 44. The only other internal barrier in the air flow path is the built in mechanical check valve within Pump Assembly 97. Thus, any moisture formed by condensation past Gore Membrane 44 will not be physically trapped inside Alcohol Monitor 100. One skilled in the art will recognize that these techniques may be applied to other types of devices that have an internal air flow path where moisture condensation poses a problem.

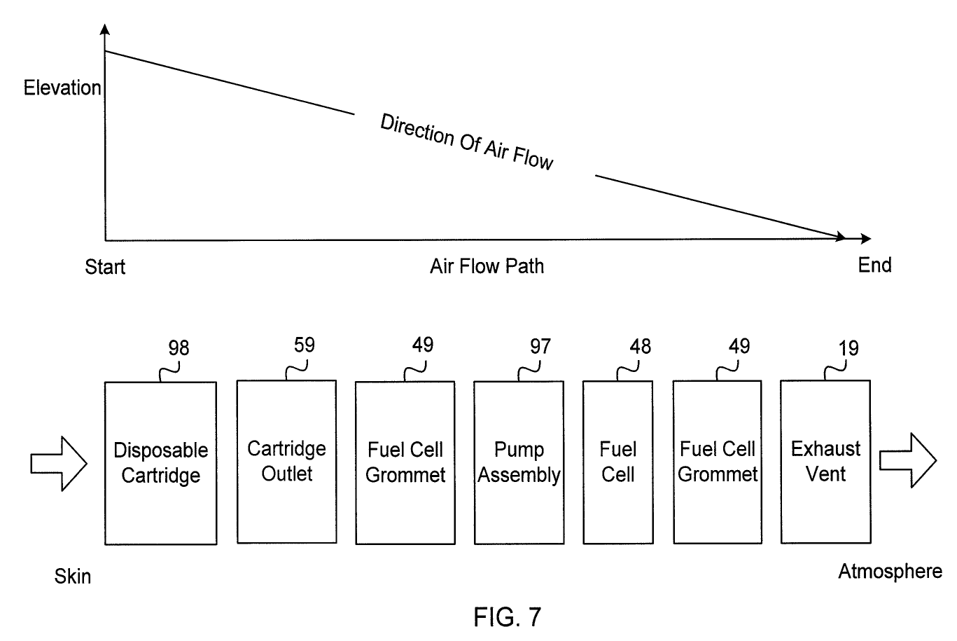

Referring now to FIG. 7, the air flow path of the present invention has been designed so that gravity helps to drain out of Alcohol Monitor 100 any moisture that forms therein. As Alcohol Monitor 100 is situated on the limb of the subject, air vapor is brought into Alcohol Monitor 100, into Disposable Cartridge 98, and through Cartridge Outlet 59 in Inner Housing 18 from a position located on the top side of the Disposable Cartridge 98. As the air passes through Alcohol Monitor 100, it works its way towards the bottom side of Alcohol Monitor 100 through Fuel Cell Grommet 49, through Pump Assembly 97, through Fuel Cell 48, through Fuel Cell Grommet 49, and out Exhaust Vent 19, located at the bottom of Inner Housing 18, and into the outside ambient air. Any water droplets that may form within Alcohol Monitor 100 will, by gravity, be drawn downward, from the top side of Alcohol Monitor 100 to the bottom side, and drained out of Sample Exhaust Hole. Therefore, Alcohol Monitor 100 of the current invention must now be oriented on the limb of the subject with a top side oriented up and a bottom side oriented down when the subject is in a standing or walking position. Water may collect in Alcohol Monitor 100 when the subject is lying down, but upon standing, any water droplets formed will begin to drain down and out of Alcohol Monitor 100 due to the force of gravity acting upon the orientation of the air flow path.

Though the invention has been described in terms of its application to a continuous blood alcohol monitoring device, such as Alcohol Monitor 100, one skilled in the art will recognize that the scope of the invention is not so limited. The present invention is applicable to any device that is attached to the body for the purpose of capturing insensible perspiration for analysis. The air coming out of the body is warm and moist and susceptible to water condensation once inside the analysis device. By lowering the humidity level of the air sample before processing it through the analysis device the chance of producing moisture through condensation is reduced. The air flow path through the analysis device should be as open as possible with as limited a number of chambers as possible, which may tend to collect and entrap condensed water. If water droplets do form, the droplets need to be processed through the system before they get a chance to create a pool of water which may interfere with sensor readings or damage device components. Designing the air path flow so that gravity will assist in draining any water droplets formed out of the analysis device will help mitigate the potential damage caused by water to the internal workings of the device and interference with sensor readings.

Having described the present invention, it will be understood by those skilled in the art that many changes in construction and circuitry and widely differing embodiments and applications of the invention will suggest themselves without departing from the scope of the present invention.

What is claimed is:

1. A method for controlling moisture within a device having an air flow path there through, the method comprising the steps of:
(a) moving an air vapor sample through an inlet opening into a sample collection chamber of the device;
(b) moving an ambient air sample through a vent into said sample collection chamber, said air vapor sample and said ambient air sample forming a combined air sample in said sample collection chamber, wherein said combined air sample has a lower humidity than said air vapor sample;
(c) moving said combined air sample into a sensor along the air flow path; and
(d) moving said combined air sample from said sensor to an exhaust vent, wherein any moisture formed by condensation past said inlet opening along the air flow path to said exhaust vent is not physically trapped within the device.

2. The method according to claim 1 further comprising the steps of:
placing the device on a limb of a subject prior to said moving steps; and
moving said air vapor sample from a surface of a subject's skin through said inlet opening and through a hydrophobic membrane, wherein said hydrophobic membrane prevents water from said air vapor sample from entering the air flow path.

3. The method according to claim 2 wherein said moving steps are performed with a pump in communication with the air flow path.

4. The method according to claim 1 wherein the device is a transdermal blood alcohol monitor and said sensor is a fuel cell.

5. The method according to claim 4 further comprising the steps of:
performing a reading on said combined air sample moved into said fuel cell, said performing step further comprising the steps of:
moving said air vapor sample across a face of said fuel cell; and
generating by said fuel cell a transdermal alcohol concentration reading.

6. A device, having an air flow path there through with moisture control features, that performs readings on air samples, the device comprising:
a body;
a sample collection chamber within said body;
an inlet opening into said sample collection chamber for receiving an air vapor sample;
a vent leading into said sample collection chamber for receiving an ambient air sample;
an air flow path through said body;
a sensor within said body located along said air flow path; and
an exhaust vent in said body located at the end of said air flow path;
wherein said air vapor sample and said ambient air sample received in said collection chamber form a combined air sample, wherein said combined air sample has a lower humidity than said air vapor sample, and said combined air sample is moved through said sensor and out of said exhaust vent, wherein any moisture formed by condensation past said inlet opening along said air flow path is not physically trapped within the device.

7. The device according to claim 6 wherein the device is a transdermal blood alcohol monitor, said sensor is a fuel cell, and said air vapor sample is drawn from a surface of a subject's skin through a hydrophobic membrane located between said inlet opening and said sample collection chamber, wherein said hydrophobic membrane prevents water from said air vapor sample from entering the air flow path.

8. The device according to claim 7 wherein said fuel cell generates transdermal alcohol concentration readings when said combined air sample is moved through said fuel cell.

9. The device according to claim 6 further comprising:
an attachment means for attaching the device to a limb of a subject.

10. The device according to claim 6 further comprising:
at least one moisture drain in a bottom side of the device for draining water out of the device that may collect within the device.

11. The device according to claim 6 wherein said sample collection chamber, said inlet opening, said vent, and said hydrophobic membrane form a disposable cartridge which snaps into said body, and may be removed and replaced from time-to-time.

12. A method for controlling moisture within a device having an air flow path there through, the method comprising the steps of:
(a) orienting the device so that a bottom side faces down toward the ground, and a top side faces up and away from the ground;
(b) moving an air vapor sample through an inlet opening into a sample collection chamber;
(c) moving an ambient air sample through a vent into said sample collection chamber, wherein said air vapor sample and said ambient air sample form a combined air sample, wherein said combined air sample has a lower humidity than said air vapor sample;
(d) moving said combined air sample from said sample collection chamber out of an outlet located toward said top side of the device and moving said combined air sample through a sensor in a generally downward direction through the air flow path in the device; and
(e) moving said combined air sample out of an exhaust vent located towards said bottom side of the device;
wherein any moisture formed by condensation within the air flow path is drawn by gravity downward through the air flow path and out of the device.

13. The method according to claim 12 wherein said orienting step further comprises the step of:
placing the device on a limb of a subject so that when said subject is in a standing or walking position, said bottom side of the device faces down toward the ground, and said top side of the device faces up and away from the ground.

14. The method according to claim 12 wherein the device is a transdermal blood alcohol monitor, said sensor is a fuel cell, and said air vapor sample is drawn from a surface of a subject's skin and through a hydrophobic membrane, wherein said hydrophobic membrane prevents water from said air vapor sample from entering the air flow path.

15. The method according to claim 14 further comprising the steps of:
performing a reading on said combined air sample moved into said fuel cell, said performing step further comprising the steps of:
moving said combined air sample through said fuel cell; and
generating by said fuel cell a transdermal alcohol concentration reading.

16. The method according to claim 12 further comprising the step of:
draining water out of the device that may collect within the device through at least one moisture drain located in a bottom side of the device.

17. The method according to claim 12 further comprising the step of:
removing and replacing a disposable cartridge from the device, wherein said disposable cartridge is comprised of said sample collection chamber, said inlet opening, said vent, and said hydrophobic membrane.

18. A device, having an air flow path there through with moisture control features, that performs readings on air samples, the device comprising:
a body having a bottom side that faces down toward the ground and a top side that faces up and away from the ground;
a sample collection chamber within said body;
an inlet opening in said sample collection chamber;
an exhaust vent located towards said bottom side of said body; and
an air flow path connecting said inlet opening to said sample collection chamber and to said exhaust vent;
wherein the air samples are moved into the device through said inlet opening, through said sample chamber, and out of said vent along said air flow path in a generally downward direction from said inlet opening to said exhaust vent, wherein any moisture formed by condensation within said air flow path is drawn by gravity downward through said air flow path and out of the device.

19. The device according to claim 18 further comprising:
an attachment means for attaching the device to a limb of a subject.

20. The device according to claim 18 wherein the device is a transdermal blood alcohol monitor, and the air samples are drawn from a surface of a subject's skin.

21. The device according to claim 20 further comprising:
a fuel cell, wherein the air samples are moved through said fuel cell, and said fuel cell generates transdermal alcohol concentration readings.

22. The device according to claim 18 further comprising:
a hydrophobic membrane located between said inlet opening and said sample collection chamber;
wherein any moisture formed by condensation past said hydrophobic membrane along said air flow path is not physically trapped within the device.

23. The device according to claim 22 wherein said sample collection chamber, said inlet opening, said vent, and said hydrophobic membrane form a disposable cartridge which snaps into said body, and may be removed and replaced from time-to-time.

24. The device according to claim 18 further comprising:
a vent in said collection chamber, wherein ambient air samples are drawn into said sample collection chamber through said vent and mixed with the air samples forming a combined air samples, wherein said combined air samples have a lower humidity than the air samples.

25. The device according to claim 18 further comprising:
at least one moisture drain in a bottom side of the device for draining water out of the device that may collect within the device.
